(19) United States
(12) Patent Application Publication
(10) Pub. No.: US 2018/0027907 A1
(43) Pub. Date: Feb. 1, 2018

(54) AROMATHERAPY SCARF WITH POCKET POUCH

(71) Applicant: Danielle A. Singer, Overland Park, KS (US)

(72) Inventor: Danielle A. Singer, Overland Park, KS (US)

(21) Appl. No.: 15/639,201

(22) Filed: Jun. 30, 2017

Related U.S. Application Data
(60) Provisional application No. 62/368,579, filed on Jul. 29, 2016.

(51) Int. Cl. A41D 27/20; A41B 1/08; A61L 9/12

(52) U.S. Cl. CPC A41D 27/207; A61L 9/12; A41B 1/08; A41D 27/20; A61L 9/04

(57) ABSTRACT

An aromatherapy garment can be donned by a user and removably receives an aromatherapy element. The garment includes a garment layer and opposed pocket flaps fixed relative to the garment layer. The pocket flaps cooperate to at least partly define a pocket that removably receives the aromatherapy element. The pocket flaps extend from corresponding locations adjacent pocket ends toward one another to at least partly cover the pocket and retain the aromatherapy element when the aromatherapy element is received therein. The pocket flaps cooperatively define an access opening therebetween that permits insertion and removal of the aromatherapy element relative to the pocket.

### RELATED APPLICATION

This application claims the benefit of U.S. Provisional Application Ser. No. 62/368,579, filed Jul. 29, 2016, entitled AROMATHERAPY SCARF WITH POCKET POUCH, which is hereby incorporated in its entirety by reference herein.

### BACKGROUND

### 1. Field

The present invention relates generally to garments. More specifically, embodiments of the present invention concern an aromatherapy garment used to dispense an aromatherapy substance for the wearer.

### 2. Discussion of Prior Art

Aromatherapy involves the use of aromatic and/or medicinal vapors (e.g., from essential oils) for healing, relaxation, mood enhancing, and grounding. Oils can be absorbed via the skin to cleanse, nourish, and rejuvenate the body. Such oils can also be used to heal burns, cuts, or other skin damage. Oil vapors can also enter the olfactory system to provide psychological and physiological benefits. Most aromatherapy substances include plant-based formulations extracted from leaves, flowers, stems, seeds, bark, or roots. Several plants are known to diminish symptoms of physical ailments such as motion sickness, nausea, and headaches. Various garments, such as scarves, have been adapted to carry aromatherapy substances.

However, conventional aromatherapy garments have multiple deficiencies. For example, known garments have a carrier element (such as a pad) that absorbs and releases an aromatherapy substance. While prior art garments have an opening to receive and carry a pad, the garments allow the pad to be inadvertently dropped from the opening and lost.

### SUMMARY

The following brief summary is provided to indicate the nature of the subject matter disclosed herein. While certain aspects of the present invention are described below, the summary is not intended to limit the scope of the present invention.

Embodiments of the present invention provide an aromatherapy garment that does not suffer from the problems and limitations of the prior art garments set forth above.

A first aspect of the present invention concerns an aromatherapy garment operable to be donned by a user and to removably receive an aromatherapy element. The aromatherapy garment broadly includes a garment layer and opposed pocket flaps. The garment layer is configured to cover at least part of the user when the aromatherapy garment is donned by the user. The pocket flaps are fixed relative to the garment layer and extend along a length direction. The pocket flaps cooperate to at least partly define a pocket with opposite pocket ends spaced along the length direction, with the pocket being configured to removably receive the aromatherapy element. The pocket flaps extend from corresponding locations adjacent the pocket ends toward one another along the length direction to at least partly cover the pocket and retain the aromatherapy element when the aromatherapy element is received therein. The pocket flaps cooperatively define an access opening therebetween that permits insertion and removal of the aromatherapy element relative to the pocket.

A second aspect of the present invention concerns an aromatherapy garment operable to be donned by a user. The aromatherapy garment broadly includes a garment layer, opposed pocket flaps, and an aromatherapy element. The garment layer is configured to cover at least part of the user when the aromatherapy garment is donned by the user. The pocket flaps are fixed relative to the garment layer and extend along a length direction. The pocket flaps cooperate to at least partly define a pocket with opposite pocket ends spaced along the length direction. The aromatherapy element is operable to deliver an aromatherapy scent to the user. The aromatherapy element is removably received in the pocket and extends between the pocket ends. The pocket flaps extend from corresponding locations adjacent the pocket ends toward one another along the length direction to at least partly cover and retain the aromatherapy element. The pocket flaps cooperatively define an access opening therebetween that permits insertion and removal of the aromatherapy element relative to the pocket.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the present invention will be apparent from the following detailed description of the embodiments and the accompanying drawing figures.

### BRIEF DESCRIPTION OF THE DRAWING FIGURES

Preferred embodiments of the invention are described in detail below with reference to the attached drawing figures, wherein:

FIG. 1 is a front perspective of an aromatherapy garment constructed in accordance with a first embodiment of the present invention, wherein the garment includes a scarf, a pouch attached to the scarf, and an aromatherapy pad retained within the pouch;

FIG. 2 is a rear perspective of the aromatherapy garment shown in FIG. 1;

FIG. 3 is a fragmentary front elevation of the aromatherapy garment shown in FIGS. 1 and 2;

FIG. 4 is a fragmentary rear elevation of the aromatherapy garment shown in FIGS. 1-3;

FIG. 5 is a fragmentary top view of the aromatherapy garment shown in FIGS. 1-4, with the opposite bottom view being a mirror image thereof;

FIG. 6 is a fragmentary side view of the aromatherapy garment shown in FIGS. 1-5, with the opposite side view being a mirror image thereof;

FIG. 7 is an enlarged fragmentary perspective of the aromatherapy garment shown in FIGS. 1-6, showing the pouch mounted to a side section of the scarf;

FIG. 8 is an enlarged fragmentary perspective of the aromatherapy garment similar to FIG. 7, but showing the pouch opened to permit removal of the aromatherapy pad;

FIG. 9 is a fragmentary cross section of the aromatherapy garment taken along line 9-9 in FIG. 3, showing the aromatherapy pad retained within the pouch;

FIG. 10 is a fragmentary front elevation of an aromatherapy garment constructed in accordance with a second embodiment of the present invention;

FIG. 11 is a fragmentary rear elevation of the aromatherapy garment shown in FIG. 10;

FIG. 12 is a fragmentary top view of the aromatherapy garment shown in FIGS. 10 and 11, with the opposite bottom view being a mirror image thereof;

FIG. 13 is a fragmentary side view of the aromatherapy garment shown in FIGS. 10-12, with the opposite side view being a mirror image thereof;

FIG. 14 is an enlarged fragmentary perspective of the aromatherapy garment shown in FIGS. 10-13, showing the pouch mounted to a side section of the scarf;

FIG. 15 is an enlarged fragmentary perspective of the aromatherapy garment similar to FIG. 14, but showing the pouch opened to permit removal of the aromatherapy pad;

FIG. 16 is a perspective of an aromatherapy garment constructed in accordance with a third embodiment of the present invention, wherein the garment includes a shirt, a pouch, and an aromatherapy pad; and

FIG. 17 is an enlarged fragmentary perspective of the aromatherapy garment shown in FIG. 16, showing the pouch attached to an interior sleeve surface of the shirt.

The drawing figures do not limit the present invention to the specific embodiments disclosed and described herein. The drawings are not necessarily to scale, emphasis instead being placed upon clearly illustrating the principles of the preferred embodiment.

### DETAILED DESCRIPTION OF THE PREFERRED EMBODIMENTS

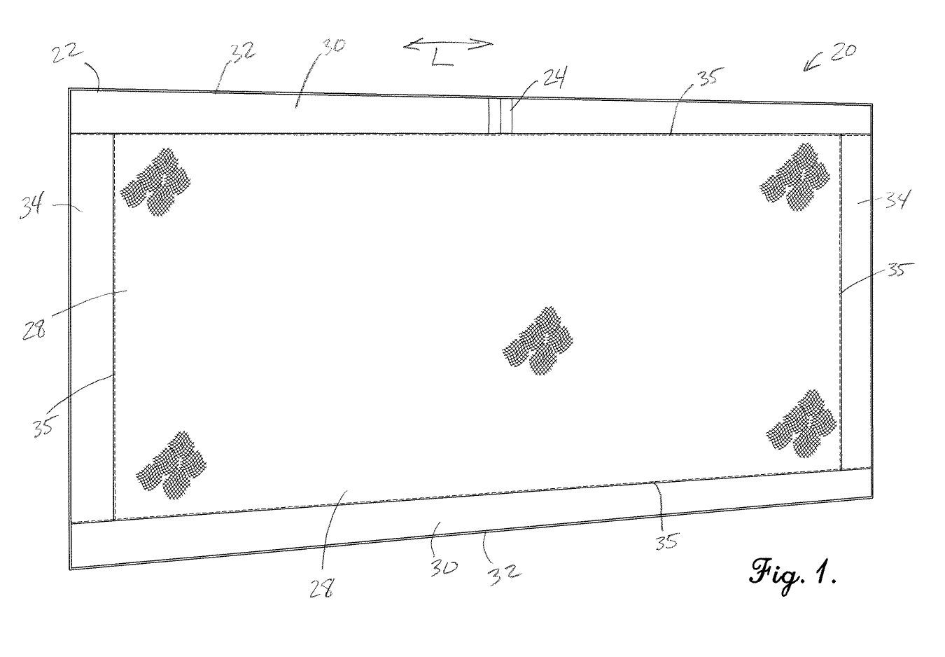

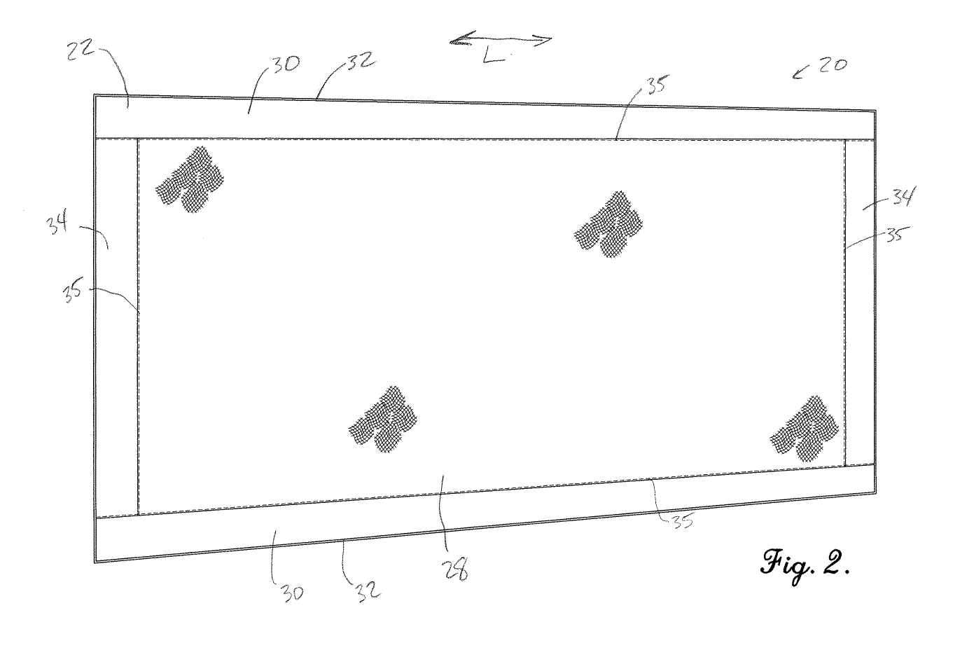

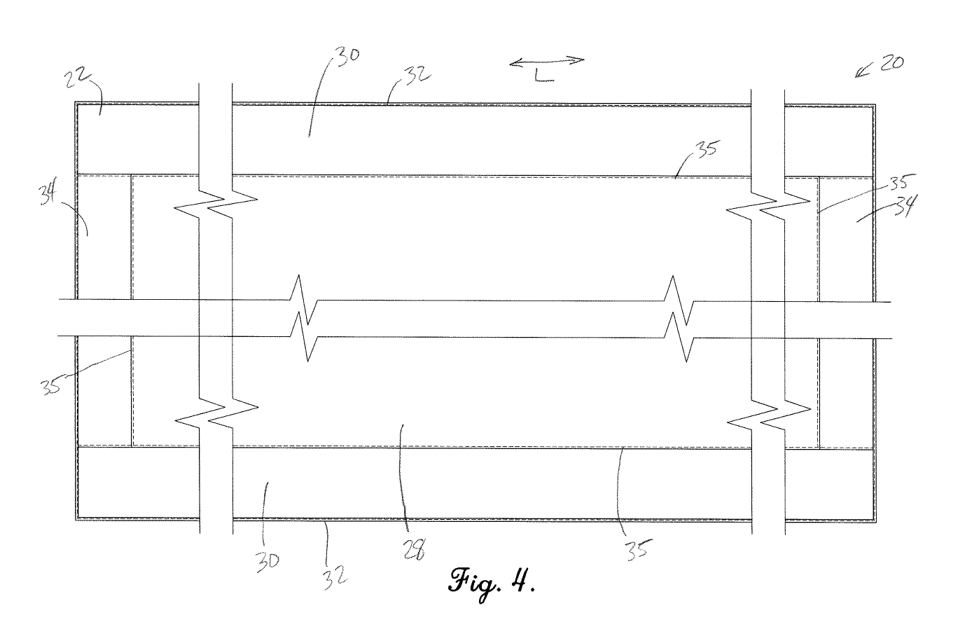

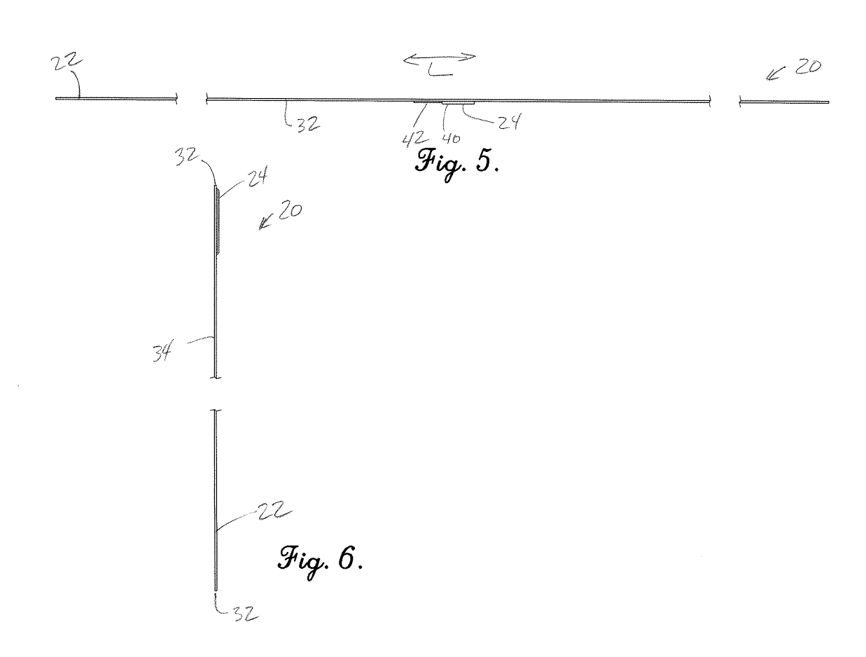

Referring to FIGS. 1-6, an aromatherapy garment 20 comprises an aromatherapy scarf that is operable to be donned by a user and includes an aromatherapy element. In the usual manner, the scarf can be wrapped around the user in a variety of configurations. For instance, it will be appreciated that the scarf may be positioned around the user's neck (not shown) so that the aromatherapy element is directly below the user's chin (e.g., so that the user's face is adjacent and directly exposed to the element).

In general, the aromatherapy system allows targeted placement of an aromatherapy substance (not shown) in close proximity to the olfactory system of the wearer to provide continuous, stimulating, relaxing, therapeutic and/or medicinal effects desired by the wearer.

The scarf can be worn around the neck, shoulders, or waist of the user, and is preferably maintained by selectively replacing and/or reusing aromatherapy pads. The depicted scarf is long and rectangular in shape, and can be worn as a scarf, shawl, or used as a blanket. Aromatic vapors from the aromatic substance are diffused through the air at normal room temperature.

Although the aromatherapy element is integrated into the depicted scarf, it will be understood that the element could be provided as part of an alternative garment (e.g., a shawl, wrap, cloak, hat, shirt, etc.). The illustrated garment 20 preferably includes a scarf 22, a pouch 24, and an aromatherapy pad 26.

The depicted scarf 22 is configured to cover at least part of the user when donned. The scarf 22 preferably includes a scarf body 28, side sections 30 that form opposite side margins 32 of the scarf 22, and end sections 34. The body 28 has an elongated, unitary shape and presents opposite sides. The body 28 is preferably formed by a single layer of material, although the body could have multiple layers.

Each side section 30 and end section 34 preferably comprises a single piece of material that is folded onto itself to form a pair of overlapping layers. The side sections 30 and end sections 34 are fixed to the body 28 by sewing the sections 30,34 thereto along seams 35. The resulting scarf 22 preferably has an elongated, rectangular shape. It will also be appreciated that the scarf could have an alternative geometrical shape (e.g., where the scarf is square or circular) for aesthetic and/or functional reasons.

The scarf 22 preferably includes a flexible fabric material such as silk, cotton, lyocell, wool, or a combination thereof (e.g., a lyocell and wool jersey blend). It is also within the ambit of the present invention where the scarf includes one or more alternative materials (such as an alternative fabric). In various embodiments, the fabric material can include a mixture of both regular fabric threads and stretch fabric threads.

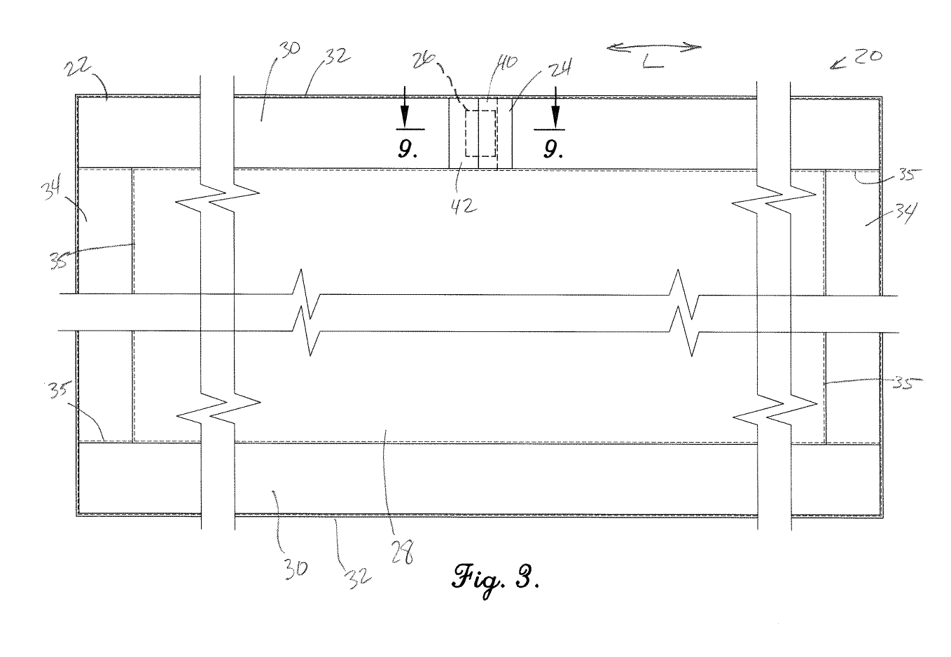

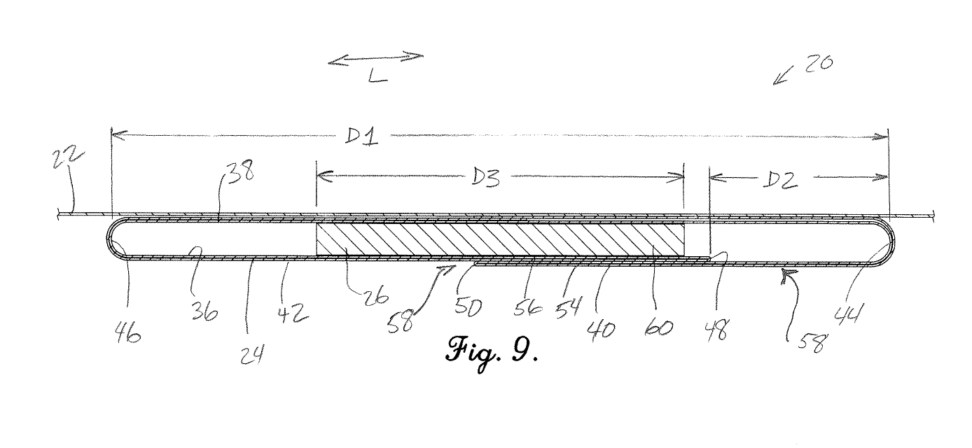

Turning to FIGS. 3 and 7-9, the pouch 24 preferably defines a pocket 36 that is configured to removably receive and secure the pad 26 while allowing the pad 26 to emit an aromatherapy substance outside of the pouch 24 in the form of liquid and/or vapor. The depicted pouch 24 includes a pouch base 38, an overlapping pocket flap 40, and an underlapping pocket flap 42 that are integrally formed with one another to form a unitary pouch structure (see FIG. 9). The pocket flaps 40,42 cooperate to at least partly define the pocket 36 and opposite pocket ends 44,46, with the ends 44,46 being spaced along a length direction L and defining a pocket length dimension D1 measured along the length direction L (see FIG. 9).

The pocket flaps 40,42 extend from corresponding locations adjacent the pocket ends 44,46 toward one another along the length direction L to at least partly cover the pocket 36 and retain the aromatherapy pad 26 when the aromatherapy pad 26 is received therein. In the illustrated embodiment, the pocket flaps 40,42 preferably enclose the corresponding pocket ends 44,46 and extend from the pocket ends 44,46 toward each other to present respective flap edges 48,50 (see FIGS. 8 and 9). However, in some alternative embodiments, one or both of the pocket flaps 40,42 could extend from locations spaced from the respective pocket end 44,46.

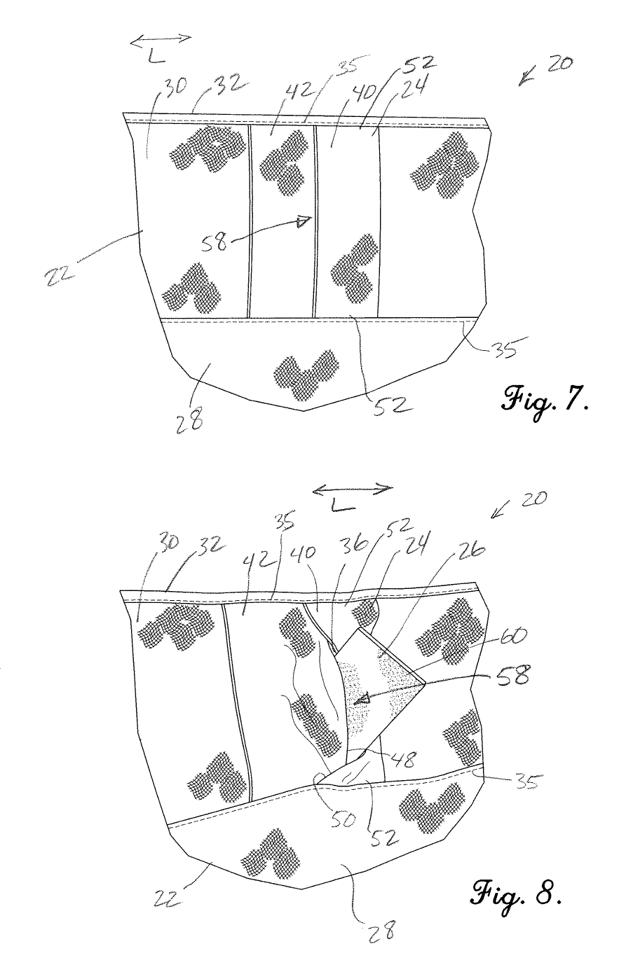

In the illustrated embodiment, the pouch 24 presents opposite side margins 52 that extend along the length direction L (see FIGS. 7 and 8). The pouch 24 is preferably fixed to the scarf 22 along the opposite side margins 52 by corresponding seams 35 (see FIGS. 7 and 8).

The depicted pocket flaps 40,42 preferably overlap one another to define overlapped flap portions 54,56 in a normal closed condition (see FIGS. 7 and 9). The pocket flaps 40,42 can be selectively moved out of the closed condition and away from each other to temporarily expand the access opening 58 in an open condition (see FIG. 8). The pocket flaps 40,42 can be selectively returned to the closed condition.

The pouch 24 presents an access opening 58 defined between the pocket flaps 40,42 (see FIGS. 7-9). The access opening 58 permits insertion and removal of the aromatherapy pad 26 relative to the pocket 36 (see FIG. 8). In the illustrated embodiment, the access opening 58 presents an opening length dimension D2 measured along the length direction L (see FIG. 9). The flap edge 48 and the pocket end 44 cooperatively define the opening length dimension D2 (see FIG. 9).

The opening length dimension D2 is preferably less than about one half the length of the pocket length dimension D1. The opening length dimension D2 is also preferably less than about one half the length of a pad length dimension D3. However, it will be appreciated that the opening length dimension D2 could fall outside one or both of these dimensional ranges without departing from the scope of the present invention.

It will also be appreciated that the configuration of pocket flaps 40,42 permits the pouch 24 to be devoid of any removable fasteners (such as a button, snap, hook-and-loop material, zipper fastener, etc.). Although the pouch 24 is preferably devoid of such fasteners, one or more removable fasteners could be used to removably secure the pocket flaps to one another or to removably secure one or both of the pocket flaps relative to the scarf 22.

However, the pocket flaps 40,42 could be alternatively configured to removably secure the pad 26 without departing from the scope of the present invention. Although the pocket flaps 40,42 preferably overlap one another, for some aspects of the present invention the pocket flaps could be in a nonoverlapping configuration while being operable to secure the pad 26. In one such alternative embodiment, the pocket flaps could extend from the respective pocket ends 44,46 toward one another without overlapping.

In another alternative embodiment, one or both of the pocket flaps could extend from a location spaced between the pocket ends toward the other one of the pocket flaps (e.g., where the pocket ends are at least partly open and exposed). For instance, one or both of the pocket flaps could comprise a unitary strip of material that does not enclose the corresponding pocket end.

Each pocket flap 40,42 extends between the pocket ends 44,46 and is fixed to the scarf 22 along the side margins 52 of the pouch 24. Although the pocket flaps 40,42 are preferably provided as part of the depicted pouch 24, the pocket flaps 40,42 could be alternatively provided with the scarf 22. For instance, the pocket flaps could be separated from one another (e.g., where the scarf 22 is devoid of the pouch base). In such an alternative embodiment, the pocket flaps could be sewn or otherwise fixed relative to the scarf 22. However, it will be appreciated that the pocket flaps could be integrally formed as part of the scarf 22 (e.g., where the scarf includes one or more folds to form at least one of the pocket flaps).

The pouch 24 preferably includes a fabric material such as silk, cotton, lyocell, wool, or a combination thereof (e.g., a lyocell and wool jersey blend). However, it is within the ambit of the present invention where the pouch 24 includes one or more alternative materials (such as an alternative fabric). In various embodiments, the fabric material can include a mixture of both regular fabric threads and stretchable elastic threads.

Although the depicted pocket flaps 40,42 comprise a flexible fabric material, it will be appreciated that one or both of the pocket flaps could be formed of another flexible material (such as a rubber material or another elastomer) or a substantially rigid material (such as synthetic resin material or a metallic material, e.g., aluminum).

The aromatherapy pad 26 is preferably configured to deliver an aromatherapy substance to the user (in the form of liquid and/or vapor). The illustrated pad 26 includes a unitary pad body 60 and the aromatherapy substance (not shown). The pad body 60 presents a generally rectangular shape but could be variously shaped without departing from the scope of the present invention. The pad body 60 preferably includes a non-toxic absorbent material, such as felt, cotton, leather, synthetic resin (e.g., in the form of an open-cell foam), or any combination thereof.

The aromatherapy substance can include one or more of various compounds (in liquid and/or vapor form), such as an essential oil, perfume, or another scented liquid. In the usual manner, oils or perfumes can enter the body of the user via the user's skin or olfactory system.

The pad body 60 is preferably configured to absorb the substance when the substance is applied to one or more surfaces of the pad body 60. The pad body 60 can be partly or completely saturated with the substance (e.g., to control the intensity and/or duration of substance discharge from the pad 26).

It will be appreciated that multiple pads 26 can be used to change, modify, or combine various scents. The pads 26 are preferably replaceable and reusable for different aromatherapy protocols. For some aspects of the present invention, the scarf 22 may be worn without the pad 26 on occasions (e.g., when a scent is not desired). Thus, the scarf 22 is operable as a dual-purpose fashion accessory and aromatherapy garment.

While the aromatherapy substance is preferably absorbed by the pad body 60, the substance could also be provided separately from the pad body 60. For instance, dried herbs such as lavender and potpourri can be inserted into the pouch 24 (either with or without the pad body 60). It will also be understood that elements (e.g., leaves, flowers, stems, seeds, bark, roots, or combinations thereof) of various other plant species could be inserted into the pouch 24 (e.g., to provide a desired scent). Also, for some aspects of the present invention, the scarf could be devoid of the aromatherapy pad.

The depicted pad 26 is removably received in the pocket and extends between the pocket ends 44,46. In the illustrated embodiment, each pocket flap 40,42 at least partly overlaps the aromatherapy pad 26. However, the garment 20 could be configured so that only one of the pocket flaps overlaps the pad 26 when the pad 26 is received in the pocket 36.

The aromatherapy pad 26 presents the pad length dimension D3, which is measured along the length direction L. The pad length dimension D3 is preferably greater than the opening length dimension D2. The pad length dimension D3 is also preferably less than half of the pocket length dimension D 1. However, the pad 26 could be alternatively sized (e.g., with respect to various features of the pouch 24).

In use, one or more of pad body 60 can be selectively saturated with an aromatherapy substance and inserted into the pouch 24. The one or more pads 26 are inserted by moving the pocket flaps 40,42 away from each other from the closed condition to the open condition to temporarily expand the access opening 58 (see FIG. 8). Once the one or more pads 26 are fully inserted into the pocket 36, the pocket flaps 40,42 can be returned to the closed condition.

With the one or more pads 26 inserted in the pocket 36 of the pouch 24, the closed pocket flaps releasably retain the one or more pads. To remove one or more pads 26 from the pouch 25, the pocket flaps 40,42 are shifted away from each other from the closed condition to the open condition so as to expand the access opening 58 (see FIG. 8). Once the one or more pads 26 are removed, the pocket flaps 40,42 can be returned to the closed condition.

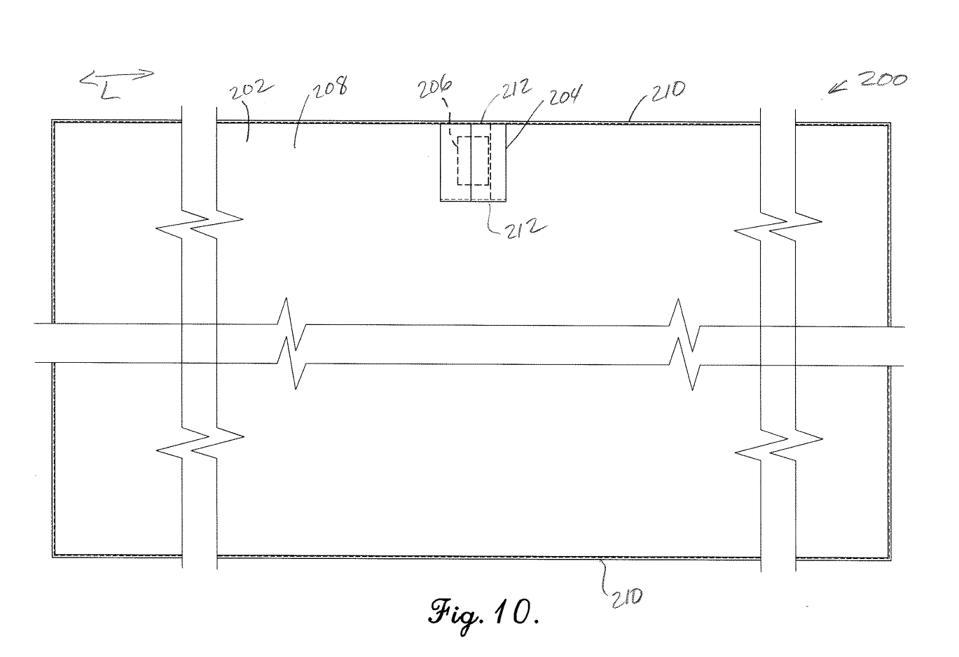

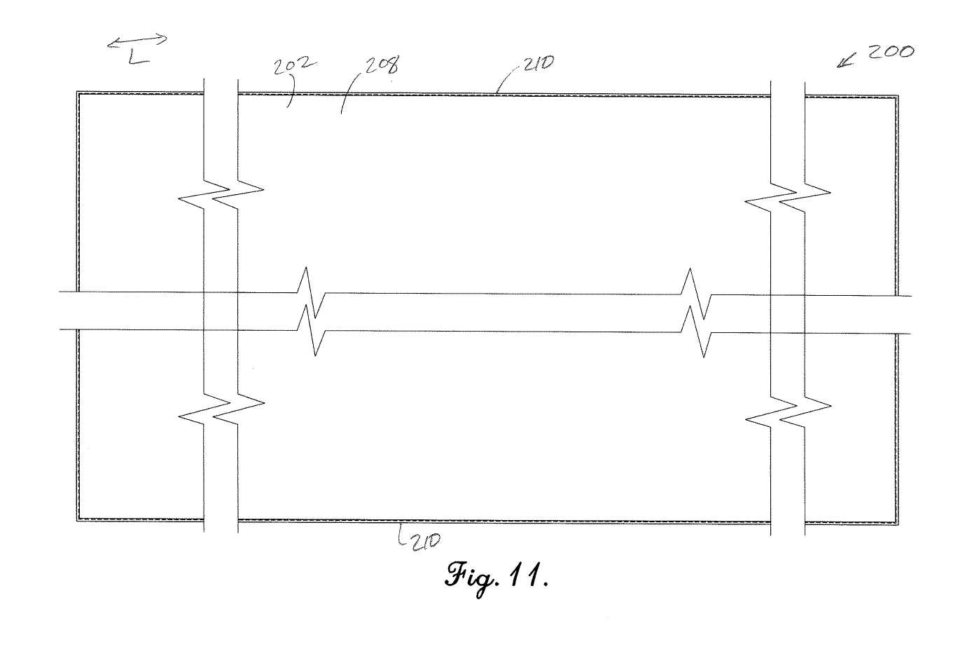

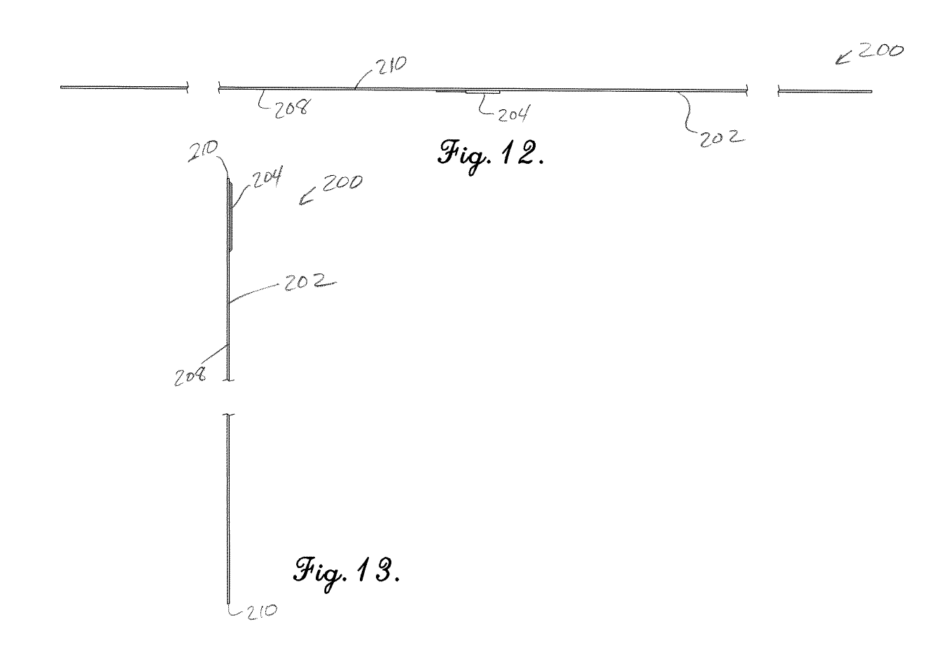

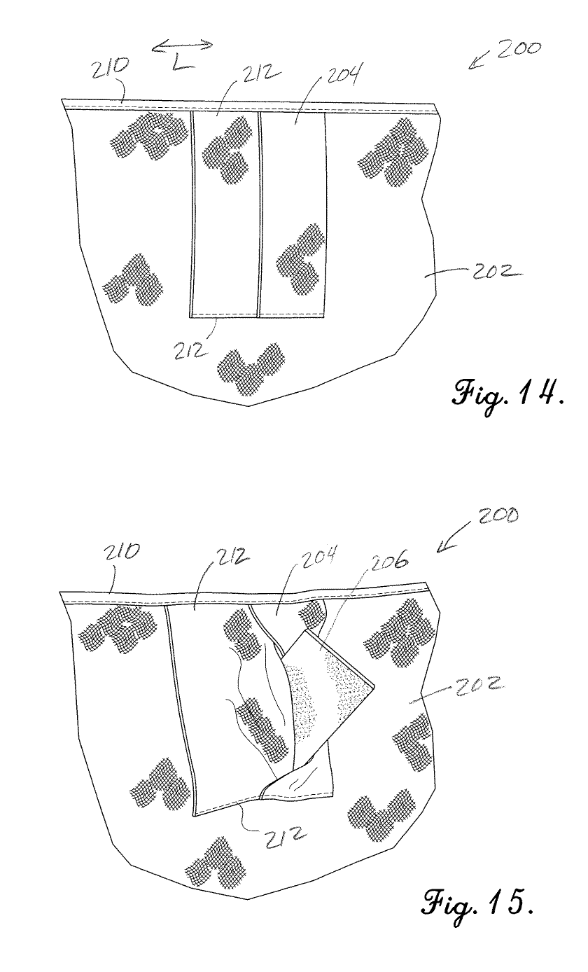

Turning to FIGS. 10-17, alternative preferred embodiments of the present invention are depicted. For the sake of brevity, the remaining description will focus primarily on the differences of these alternative embodiments from the preferred embodiment described above.

Initially turning to FIGS. 10-15, an alternative garment 200 is constructed in accordance with a second embodiment of the present invention. The alternative garment 200 includes an alternative scarf 202, a pouch 204, and an aromatherapy pad 206.

The scarf 202 preferably comprises a scarf body 208 that presents opposite side margins 210 of the scarf 202. The body 208 has an elongated, unitary shape and preferably formed by a single layer of material, although the body could have multiple layers.

In the illustrated embodiment, the pouch 204 presents opposite side margins 212 that extend parallel to side margins 210 along the length direction L and are preferably fixed to the scarf 202.

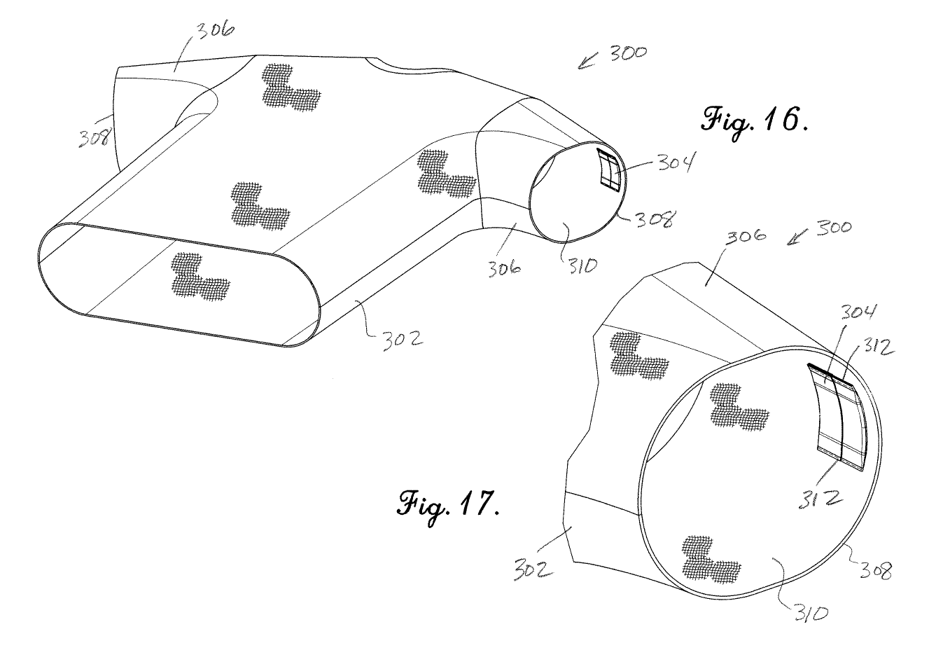

Turning to FIGS. 16 and 17, an alternative garment 300 is constructed in accordance with a third embodiment of the present invention. The alternative garment 300 includes a shirt 302, a pouch 304, and an aromatherapy pad (not shown).

The shirt 302 includes a pair of sleeves 306 that each present a distal margin 308. The pouch 304 is secured to an interior surface 310 of one of the sleeves 306 by sewing the pouch 304 along side margins 312 of the pouch 304. The pouch 304 is arranged so that the side margins 312 extend generally perpendicular to the distal margin 308 of the corresponding sleeve 306.

Although the above description presents features of preferred embodiments of the present invention, other preferred embodiments may also be created in keeping with the principles of the invention. Such other preferred embodiments may, for instance, be provided with features drawn from one or more of the embodiments described above. Yet further, such other preferred embodiments may include features from multiple embodiments described above, particularly where such features are compatible for use together despite having been presented independently as part of separate embodiments in the above description.

The preferred forms of the invention described above are to be used as illustration only, and should not be utilized in a limiting sense in interpreting the scope of the present invention. Obvious modifications to the exemplary embodiments, as hereinabove set forth, could be readily made by those skilled in the art without departing from the spirit of the present invention.

The inventor hereby states her intent to rely on the Doctrine of Equivalents to determine and assess the reasonably fair scope of the present invention as pertains to any apparatus not materially departing from but outside the literal scope of the invention as set forth in the following claims.

What is claimed is:

1. An aromatherapy garment operable to be donned by a user and to removably receive an aromatherapy element, said aromatherapy garment comprising:
a garment layer configured to cover at least part of the user when the aromatherapy garment is donned by the user; and
opposed pocket flaps fixed relative to the garment layer and extending along a length direction,
said pocket flaps cooperating to at least partly define a pocket with opposite pocket ends spaced along the length direction, with the pocket being configured to removably receive the aromatherapy element,
said pocket flaps extending from corresponding locations adjacent the pocket ends toward one another along the length direction to at least partly cover the pocket and retain the aromatherapy element when the aromatherapy element is received therein,
said pocket flaps cooperatively defining an access opening therebetween that permits insertion and removal of the aromatherapy element relative to the pocket.

2. The aromatherapy garment as claimed in claim 1,
said pocket flaps enclosing the corresponding pocket ends and extending therefrom toward one another.

3. The aromatherapy garment as claimed in claim 2,
each of said pocket flaps presenting opposite side margins extending between the pocket ends and fixed to the garment layer.

4. The aromatherapy garment as claimed in claim 2,
said pocket flaps overlapping one another, with overlapped portions of the pocket flaps cooperatively forming the access opening.

5. The aromatherapy garment as claimed in claim 4, further comprising:
a unitary pouch including the pocket flaps and a pouch base that are integrally formed with one another,
said pouch base extending between the pocket ends and cooperating with the pocket flaps to form the pocket.

6. The aromatherapy garment as claimed in claim 5,
said pouch presenting opposite side margins extending between the pocket ends and fixed to the garment layer.

7. The aromatherapy garment as claimed in claim 5,
said pouch being devoid of a releasable fastener.

8. An aromatherapy garment operable to be donned by a user, said aromatherapy garment comprising:
a garment layer configured to cover at least part of the user when the aromatherapy garment is donned by the user;
opposed pocket flaps fixed relative to the garment layer and extending along a length direction,
said pocket flaps cooperating to at least partly define a pocket with opposite pocket ends spaced along the length direction; and
an aromatherapy element operable to deliver an aromatherapy scent to the user,
said aromatherapy element being removably received in the pocket and extending between the pocket ends,
said pocket flaps extending from corresponding locations adjacent the pocket ends toward one another along the length direction to at least partly cover and retain the aromatherapy element,
said pocket flaps cooperatively defining an access opening therebetween that permits insertion and removal of the aromatherapy element relative to the pocket.

9. The aromatherapy garment as claimed in claim 8,
said pocket flaps enclosing the corresponding pocket ends and extending therefrom toward one another.

10. The aromatherapy garment as claimed in claim 9,
each of said pocket flaps presenting opposite side margins extending between the pocket ends and fixed to the garment layer.

11. The aromatherapy garment as claimed in claim 9,
said pocket flaps overlapping one another, with overlapped portions of the pocket flaps cooperatively forming the access opening.

12. The aromatherapy garment as claimed in claim 11, further comprising:
a unitary pouch including the pocket flaps and a pouch base that are integrally formed with one another, said pouch base extending between the pocket ends and cooperating with the pocket flaps to form the pocket.

13. The aromatherapy garment as claimed in claim 12,
said pouch presenting opposite side margins extending between the pocket ends and fixed to the garment layer.

14. The aromatherapy garment as claimed in claim 12,
said pouch being devoid of a releasable fastener.

15. The aromatherapy garment as claimed in claim 8,
said aromatherapy element comprising a unitary aromatherapy pad configured to receive an aromatherapy substance.

16. The aromatherapy garment as claimed in claim 15,
said pocket flaps enclosing the corresponding pocket ends and extending therefrom toward one another,
each of said pocket flaps at least partly overlapping the aromatherapy pad.

17. The aromatherapy garment as claimed in claim 16,
said pocket flaps overlapping one another to define overlying and underlying pocket flaps that extend from respective first and second ones of the pocket ends,
said underlying pocket flap presenting a flap edge, with the flap edge and the first pocket end cooperatively defining an access opening length measured along the length direction,
said aromatherapy pad presenting a pad length measured along the length direction, with the pad length being greater than the access opening length.

18. The aromatherapy garment as claimed in claim 17,
said access opening length being less than half of the pad length.

19. The aromatherapy garment as claimed in claim 18,
said pocket ends cooperatively defining a pocket length measured along the length direction,
said pad length being less than half of the pocket length.

20. The aromatherapy garment as claimed in claim 17, further comprising:
a unitary pouch including the pocket flaps and a pouch base that are integrally formed with one another,
said pouch base extending between the pocket ends and cooperating with the pocket flaps to form the pocket.
